(19) United States
(12) Patent Application Publication
(10) Pub. No.: US 2007/0117232 A1
(43) Pub. Date: May 24, 2007

(54) Method of studying interaction between immersion fluid and substrate

(76) Inventor: Roel Gronheid, Boutersem (BE)

Correspondence Address: KNOBBE MARTENS OLSON & BEAR LLP, 2040 MAIN STREET, FOURTEENTH FLOOR, IRVINE, CA, 92614, US

(21) Appl. No.: 11/593,387

(22) Filed: Nov. 6, 2006

(30) Foreign Application Priority Data
Nov. 7, 2005 (EP) EP 05447246.9

(51) Int. Cl. H01L 21/00; G01N 33/00

(52) U.S. Cl. 438/16; 438/14; 73/61.41; 73/866

(57) ABSTRACT

A method and measurement tools for studying interaction between an immersion fluid and a material in a substrate, such as, e.g., a photoresist or a topcoat or components thereof, are provided. The method comprises providing a spatially controlled contact between only part of the substrate and the immersion fluid. The latter can, e.g., be performed in a lithographic processing system. A possible way of providing spatially controlled contact is by scanning with the immersion fluid over a region of the substrate comprising the at least first material. After the contacting has been performed, analysis results are obtained for at least one of the immersion fluid or the first material in the substrate. The latter allows determination of an interaction parameter indicative of the interaction between the immersion fluid and the first material in the substrate. Such results can be used for studying dynamic leaching behaviour and for optimizing the corresponding immersion lithographic processing.

### CROSS-REFERENCE TO RELATED APPLICATIONS

This application claims the benefit under 35 U.S.C. § 119(a)-(d) of European application No. 05447246.9, filed Nov. 7, 2005, the disclosure of which is hereby expressly incorporated by reference in its entirety and is hereby expressly made a portion of this application.

### FIELD OF THE INVENTION

The preferred embodiments relate to immersion lithographic processing of substrates, e.g., in semiconductor processing. More particularly, the preferred embodiments relate to interaction between immersion fluids and substrates during immersion lithographic processing thereof.

### BACKGROUND OF THE INVENTION

In the production of today's integrated circuits, optical lithography is one of the key manufacturing techniques. In order to cope with the ongoing miniaturisation of integrated circuits or other devices and its associated problems, high numerical aperture solutions such as immersion lithographic processing are often applied.

Switching from dry lithography to immersion lithography alters the possible mechanisms for contamination in the optical path of a lithographic system. Using immersion fluid in contact with both the final optical lens of the immersion lithography system and the substrate may lead to changes in the immersion fluid, to changes in the substrate and to contamination of the immersion lithographic system, i.e., of the final lens element in the immersion hood. Changes in the substrate, e.g., the resist or the top coat, typically are caused by penetration of components of the immersion fluid into and leaching of resist components out of the substrate and may result in an altered imaging performance. Changes in the immersion fluid typically are caused by dissolution of components of the substrate, e.g., components of the resist or of a top coat layer on top of the resist, in the immersion fluid, leading to altered optical properties of the immersion fluid. The dissolution of components of the substrate, also referred to as leaching, typically also results in contamination of the final lens element. The lens contamination negatively influences the lens lifetime, which is a function of the degradation rate of contaminated lens material under typical illumination conditions and of the immersion fluid refreshment rate and dynamics in the immersion hood. Typical components that may deteriorate the lens lifetime are photo-acid generators (PAG) and quenchers, present in the resist layer. Therefore, immersion lithography system manufacturers typically provide specific leaching requirements for materials to be used with their system.

Some methods and devices for studying the interaction between the immersion fluid and the substrate have already been developed.

In Advances in Resist Technology and Processing XXII, Proceedings of SPIE 5753 (SPIE, Bellingham, Wash., 2005), Conley et al. report on a study of leaching of photo-acid generator from resist material in water. Wafers coated with resist are placed in a 2 inch Petri dish and covered with water for contact times ranging from 30 seconds to 30 minutes. It was concluded that photo-acid generator leaching occurs only upon initial contact with water.

In J. Photopolym. Sci. Technol. 17 (2004) pages 591-594, Dammel et al. describe a method for studying leaching of components from a resist in water. Leaching thereby is studied in an immersion chamber, providing room for a full wafer and an approximately 1 mm thick layer of immersion fluid over the entire wafer and an immersion fluid entrance and exit. After the wafer has been positioned in the immersion chamber, immersion fluid is injected via the immersion fluid entrance thus providing contact with the substrate. The liquid is subsequently removed and analysed, providing information on the leaching behaviour of the components of the resist. A typical liquid filling time or liquid removal time of the immersion chamber is about 3 seconds each, leading to a minimum interaction time of at least about 6 second.

EP-A-1 519 231, describes a method for immersion lithographic processing of a substrate. The method comprises controlling the exposure dose during immersion lithographic processing for correcting for photoresist dissolution. The amount of photoresist dissolution is determined using a closed loop system wherein analysis of the immersion liquid composition or the PH-value is performed for obtaining information about the amount of dissolution of the photoresist in the immersion liquid or the amount of diffusion of immersion liquid into the photoresist.

### SUMMARY OF THE INVENTION

Other techniques and systems still are required to further explore leaching effects between a substrate, e.g., resist layer, and an immersion fluid.

An improved method and improved measurement tools are provided for studying interaction between an immersion fluid and a substrate as well as to improved lithographic processing. It is an advantage of the preferred embodiments that the dynamic behaviour of interaction between a substrate and an immersion fluid can be studied, i.e., the behaviour of interaction as a function of time. Moreover, it is an advantage of the preferred embodiments that interaction between a substrate and an immersion fluid for short interaction time periods can be studied. It is also an advantage of the preferred embodiments that studying of interaction between a substrate and an immersion fluid can be performed with high controllability. It furthermore is an advantage of the preferred embodiments that interaction effects between a substrate and an immersion fluid can be studied with high accuracy.

The above advantages are achieved by a method and device according to the preferred embodiments.

The preferred embodiments relate to a method for determining an interaction parameter indicative of an interaction between an immersion fluid and a material or for studying interaction between an immersion fluid and at least a first material in a substrate suitable for lithographic processing, the method comprising providing a spatially controlled contact by providing a relative movement of a contact area between the substrate and the immersion fluid over the substrate; thereafter, obtaining analysis results of at least one of the immersion fluid or the at least a first material in the substrate; and, determining from the analysis results an interaction parameter indicative of the interaction between the immersion fluid and the at least a first material in the substrate.

It is an advantage of the preferred embodiments that also interaction in a short interaction time between the immersion fluid and the first area of a substrate suitable for lithographic processing can be studied. With a short interaction time there can be meant interaction times of less than 5 seconds, more preferably of less than 3 seconds even more preferably of less than 2 seconds, still more preferably of less than 1 second. The latter is, e.g., advantageous over methods and systems based on pouring water on a substrate and removing it, which results in a lowest controlled time-resolution of about 5 seconds. The preferred embodiments can allow the study of the dynamic interaction behaviour between the immersion fluid and the first materials, especially as leaching of, e.g., photo-acid generators saturates in short timeframes, e.g., <5 seconds, and as the contact times between immersion fluid and substrate is short, e.g., less than 1.5 seconds, in a high volume manufacturing lithography system. The results can stem from a measurement on the immersion fluid or the at least a first material in the substrate after these have been brought in the spatially controlled contact.

The contact can be a contact between the immersion fluid and only part of the substrate. Only part of the substrate can be only part of one side or surface of the substrate. The first material can comprise first components, wherein determining an interaction parameter can comprise determining an interaction parameter indicative of leaching from at least one of the first components of the at least first material in the immersion fluid. Performing an analysis can be performing an analysis of the presence of a first component in the immersion fluid. It is an advantage of preferred embodiments that the dynamic behaviour of leaching from materials suitable for lithographic processing to the immersion fluid can be studied.

The first material can be any of a photoresist or a top anti-reflective coating. The first components of the first material can be a photo-acid generator of the photoresist or components thereof. It also can be a quencher or components thereof. It furthermore also can be a residual casting solvent or components thereof. The first components can also be components of the top-anti-reflective coating material.

A physical size of the contact area, such as, e.g., the physical surface area, can be substantially constant during the providing a spatially controlled contact.

Providing a relative movement of the contact area can comprise providing a contact path by providing a relative movement between the immersion fluid and a first region of the substrate parallel to the substrate.

The method can comprise, prior to providing a spatially controlled contact, providing the substrate in an immersion lithography processing system comprising an immersion hood and providing the immersion fluid to the immersion hood, wherein providing a spatially controlled contact is performed using a relative movement between the immersion hood and the substrate. The method can comprise, prior to the obtaining analysis results, collecting the immersion fluid, e.g., from the immersion lithography processing system. It is an advantage of certain preferred embodiments that the interaction, i.e., occurring during the providing spatially controlled contact, can be performed in substantially the same conditions as the interaction during lithographic processing of the substrate. It is an advantage of certain preferred embodiments that the interaction can be studied under exposure conditions.

The substrate can further comprise at least a second region, the second region being free from materials comprising the first components, wherein providing a contact path can comprise initiating the contact path between the immersion fluid and the substrate in the at least a second region; then contacting the immersion fluid with the first region; and thereafter, ending the contact path between the immersion fluid and the substrate in the at least a second region.

The interaction time between the immersion fluid and the first material in a first region of a substrate can be selected by selecting any of a speed of relative movement or a contact path length. The interaction time also can be selected by selecting a physical size of the contact area.

Obtaining analysis results of at least one of the immersion fluid or the at least a first material in the substrate can comprise using any of mass spectroscopy, scanning electrochemical microscopy or liquid scintillation counting.

The preferred embodiments also relate to a method for setting up lithographic processing of a substrate, the method comprising selecting any of an immersion fluid, a substrate, system parameters of a lithographic processing system or processing parameters of a lithographic process based on dynamic interaction results between an immersion fluid and at least a first material in the substrate as obtained by a method for studying interaction as described above.

The preferred embodiments further relate to a measurement tool for studying the interaction between an immersion fluid and at least a first material in a substrate suitable for lithographic processing, the measurement tool comprising a means for providing a spatially controlled contact by providing a relative movement of a contact area between the substrate and the immersion fluid over the substrate. The means for providing a spatially controlled contact can comprise a substrate table and a nozzle adapted for providing immersion fluid in contact with a substrate, whereby one of the substrate table or the nozzle can be moveable in an X-direction, Y-direction or an X-Y direction.

The preferred embodiments also relate to a measurement kit for studying the interaction between an immersion fluid and at least a first material in a substrate suitable for lithographic processing according to a method for studying as described above, the measurement kit comprising the substrate having a first region comprising at least the first material suitable for lithographic processing, the first material comprising first components and having a second region being free of materials comprising the first components.

The preferred embodiments further relate to a computing device for deriving an interaction parameter for interaction between an immersion fluid and at least a first material in a substrate, the computing means comprising a means adapted for obtaining information about a spatially controlled contacting step comprising providing a relative movement of a contact area between the substrate and the immersion fluid over the substrate; a means adapted for obtaining information about a quantitative analysis of a component of the immersion fluid in the substrate or of a component of the substrate in the immersion fluid a means adapted for deriving from the obtained information a value for an interaction parameter indicative of an interaction between the immersion fluid and the at least a first material in the substrate.

The preferred embodiments also relate to a computer program product for deriving an interaction parameter for interaction between an immersion fluid and at least a first material in a substrate, the computer program product adapted for obtaining information about a spatially controlled contacting step comprising providing a relative movement of a contact area between the substrate and the immersion fluid over the substrate; obtaining information about a quantitative analysis of a component of the immersion fluid in the substrate or of a component of the substrate in the immersion fluid; and for deriving from the obtained information a value for an interaction parameter indicative of an interaction between the immersion fluid and the at least a first material in the substrate.

The preferred embodiments also relate to a machine readable data storage device storing the computer program product as described above and to the transmission of the computer program product over a local or wide area telecommunications network.

It is an advantage of preferred embodiments that the obtained results on interaction between an immersion fluid and a substrate can be used for providing material specifications.

Particular and preferred aspects of the invention are set out in the accompanying independent and dependent claims. Features from the dependent claims may be combined with features of the independent claims and with features of other dependent claims as appropriate and not merely as explicitly set out in the claims. Although there has been constant improvement, change and evolution of methods for studying leaching, the present concepts are believed to represent substantial new and novel improvements, including departures from prior practices, resulting in the provision of more efficient, stable and reliable methods for studying leaching. The preferred embodiments permit the design of improved methods and systems for lithographic processing of substrates. The above and other characteristics, features and advantages of the preferred embodiments will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a flow diagram of a method for studying interaction between a substrate and immersion fluid according to a first aspect.

FIG. 2 is a schematic illustration of a set-up as can be used for a dynamic interaction measurement in a method for studying interaction between a substrate and immersion fluid according to preferred embodiments.

FIG. 3 is a schematic illustration of an alternative set-up as can be used for a dynamic interaction measurement in a method for studying interaction between a substrate and immersion fluid according to preferred embodiments.

FIG. 4a to 4c are schematic illustrations of the relative positions of the immersion fluid, the substrate and the materials to be tested on interaction with the immersion fluid, for different steps in a method for studying interaction according to a second aspect.

FIG. 5 shows a schematic view of simulation results for the soak times of the different areas on a substrate and of the contact path for interaction between materials to be studied and an immersion fluid, as used in a method for studying interaction according to a third aspect.

FIG. 6 illustrates a schematic representation of a measurement tool for studying interaction between a material on a substrate and an immersion fluid, according to a sixth aspect.

FIG. 7 illustrates a schematic representation of a computing means as can be used for studying interaction between a material on a substrate and an immersion fluid, according to a seventh aspect.

FIG. 8 shows experimental results for a study of the interaction between a PAG anion of PAR-817 resist and immersion fluid water as obtained using a method as described in the second aspect.

FIG. 9 shows experimental results for a study of the effect of different solvents used on leaching of a PAG anion of PAR-817 , as obtained using a method as described in the second aspect.

FIG. 10a and FIG. 10b shows experimental results for a study of the effect of exposure on leaching of a PAG anion of PAR-817 (FIG. 10a) and of PAR-817 covered with a top coat (FIG. 10b), as obtained using a method as described in the second aspect.

In the different figures, the same reference signs refer to the same or analogous elements.

### DETAILED DESCRIPTION OF THE PREFERRED EMBODIMENT

The following terms are provided solely to aid in the understanding of the preferred embodiments. These definitions should not be construed to. have a scope less than understood by a person of ordinary skill in the art. In preferred embodiments, the term “substrate” can include not only the basic carrier material but also materials to be processed and typical materials applied for performing a lithographic processing step, such as for example—but not limited to—a bottom anti-reflective coating and/or a resist layer and/or a top coating. The top coating can be a protective coating and/or can be a top anti-reflective coating. The term “substrate” furthermore also can include other materials, such as, e.g., other materials used in semiconductor processing. The basic carrier material can be for example doped or undoped silicon, silicon-on-insulator substrate (SOI), gallium arsenide (GaAs), gallium arsenide phosphide (GaAsP), indium phosphide (InP), germanium (Ge), or silicon germanium (SiGe), glass or quartz substrates.

The present invention will be described with respect to particular embodiments and with reference to certain drawings but the invention is not limited thereto but only by the claims. The drawings described are only schematic and are non-limiting. In the drawings, the size of some of the elements may be exaggerated and not drawn on scale for illustrative purposes. The dimensions and the relative dimensions do not correspond to actual reductions to practice of the invention. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

The invention will now be described by a detailed description of several embodiments of the invention. It is clear that other embodiments of the invention can be configured according to the knowledge of persons skilled in the art without departing from the true spirit or technical teaching of the invention, the invention being limited only by the terms of the appended claims.

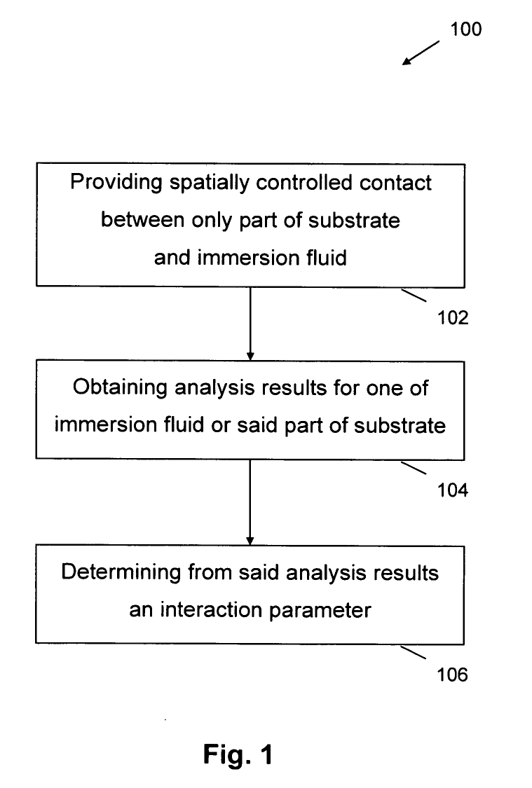

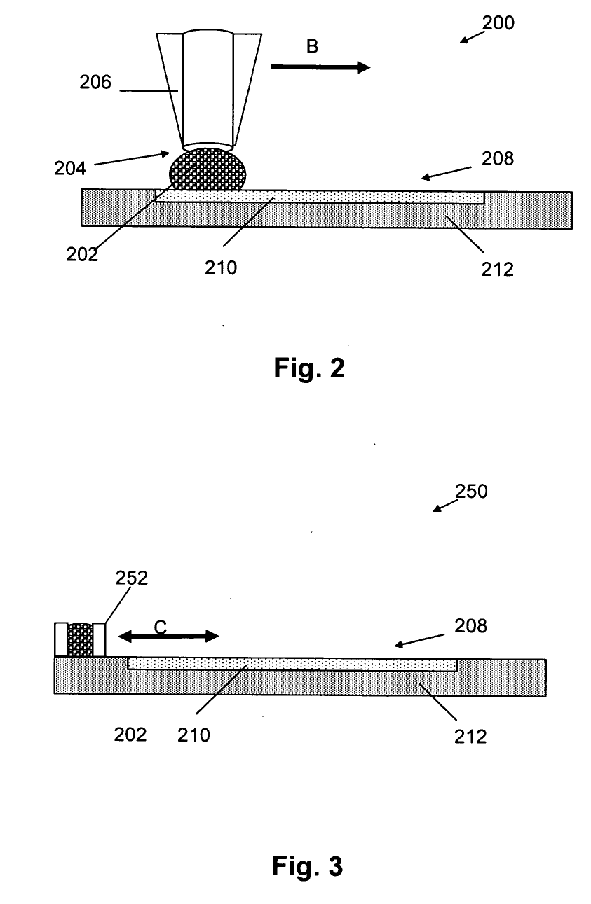

In a first aspect, a method is provided for studying interaction between an immersion fluid and materials present in a substrate suitable for lithographic processing, especially in semiconductor processing, e.g., using a semiconductor carrier and layer thereon such as a resist layer. Such materials can be, for example, material and/or components present in a resist or in a top coating such as a top anti-reflective coating. The preferred embodiments thus allow study of the interaction of an immersion fluid with materials contacted during immersion lithographic processing of a substrate. Typical steps performed in the method according to the first aspect are shown in a flow diagram of the method for studying interaction 100 in FIG. 1. The method for studying interaction 100 typically comprises providing a spatially controlled contact between the substrate and the immersion fluid by providing a relative movement of a contact area of the substrate and the immersion fluid over the substrate. With contact area, the contact area at the interface at a given time is meant. The spatially controlled contact can be a contact between the immersion fluid and only part of the substrate. The latter is performed in step 102. It can be performed by providing a relative movement between an immersion fluid and a region of the substrate, the region comprising the materials for which interaction is to be studied, i.e., a region where resist or a top coating is applied. Only a part of the substrate may contain material that is investigated. Of this part only a limited region may be exposed. The latter can be referred to as a dynamic interaction measurement. The controlled contact is then determined by the rate of movement of the immersion fluid, the surface area of the region of the substrate contacted and the physical dimensions of the immersion fluid at the contact interface. Such movement can, e.g., be performed using an immersion lithographic scanning systems, whereby at least one of the immersion hood or the substrate is moveable, in order to allow relative movement. Nevertheless, less complicated set-ups also can be used. Such less complicated set-ups can, e.g., comprise a moveable substrate stage, a means for providing and removing immersion fluid in contact with a substrate and a means for providing movement of the contact area between the substrate and the immersion fluid. A schematic illustration of a usable set-up 200 is shown in FIG. 2. An immersion fluid 202 positioned at a tip 204 of a carrier 206, such as, e.g., an immersion hood of an immersion lithographic system, and a region 208 of a substrate 212, the region 208 comprising materials 210 for which the interaction properties are to be studied, are moved relatively to each other. Relative movement can, e.g., be provided in the direction of arrow B, e.g., in a direction parallel with the surface of substrate 212. As only a limited part of the region may be exposed, the size of the carrier 206 may be less than the dimension of the area under investigation 208. An alternative example of a way of providing controlled contact can be by providing a ring shaped housing, wherein immersion fluid can be provided on the substrate, filling the ring shaped housing with an immersion fluid, shifting the ring shaped housing over the surface of the substrate and removing the fluid from the ring shaped housing. The ring 252 and the substrate 212 are thus moved relative with respect to each other, e.g., as indicated for set-up 250 in FIG. 3 by the direction of arrow C. With spatially controlled contact in step 102 there may be meant that the contact area, e.g., the physical size thereof, is controlled by the user or is automatically controlled. Furthermore, the interaction time can be controlled by the user or can be automatically controlled. Also the interaction interface at each moment of the interaction time span can be controlled by the user or be automatically controlled. Contact can be provided between the immersion fluid and only part of the substrate, meaning only part of one side of the substrate surface. With only part of the substrate there may be meant not the full substrate. Only part of the substrate also may be not the full surface of a single major side of the substrate. The latter thus excludes covering a substrate or an entire surface thereof completely with immersion fluid.

After providing a spatially controlled contact, analysis results are obtained for at least one of the immersion fluid or the area on the substrate comprising the materials for which interaction is to be studied, and which have been contacted with each other. Also part of the immersion fluid or part of the area can be used. This is indicated in step 104. Such analysis can be a qualitative analysis, or more preferably a quantitative analysis, of the presence or concentration of one or more components of the immersion fluid or part thereof in the area on the substrate or of the presence or concentration of one or more predetermined components of the materials present in the area on the substrate in the immersion fluid or part thereof. Such analysis can be performed with any suitable analysis technique. Such analysis techniques preferably are suitable to detect with a detection limit better than a few parts per billion, more preferably with a detection limit better than a few tenths of a part per billion or even better than one tenth of a part per billion and having a detection range up to at least a few hundred parts per billion, preferably up to at least a few parts per million. Typical examples of analysis techniques, the preferred embodiments not being limited thereto, are mass spectrometry, liquid chromatography-mass spectrometry (LCMS), e.g., with atmospheric pressure electrospray ionisation (AP-ESI), lyofilization, scanning electrochemical microscopy (SECM) and liquid scintillation counting (LSC). Depth profiling of the chemical composition of a substrate can be performed using for example the gradient shaving preparation (GSP) method in combination with analytical techniques such as time of flight-SIMS or fourier-transform infrared spectroscopy (FTIR). Analysis also can be performed indirectly, e.g., by evaluating lithographic processed structures using the substrate obtained after step 102. In the latter case, structures in the resist obtained after exposure and development of the exposed resist can be studied. Another indirect technique for studying the resist development rate uses a resist development analyser (RDA). The later allows studying the development rate of resist as a function of exposure. This can in principle be done for areas having a different immersion fluid contact time.

From the obtained analysis results, an interaction parameter can be determined indicative of an interaction between the immersion fluid and materials and/or components on the substrate, e.g., the resist, a topcoat, components of the resist or components of the top coat, as indicated by step 106. The interaction parameter can be, e.g., a value related to the amount of leached material of a specific component in the immersion fluid per unity of surface contact area, a value related to the amount of an immersion fluid component that has diffused in the materials Under study per unity of surface contact area, a value related to the amount of component that has leached or diffused per unity of surface contact area and per unit of interaction time for a given interaction time, etc. For determining these values, typically information about step 102 can be used, e.g., the total surface area of the contact path or contact location, the scan rate or contact time, the physical size and/or the physical shape of the immersion fluid at the interaction interface between the immersion fluid and the substrate possibly at different times of the interaction or throughout the interaction, the applied immersion fluid volume, the distance between the immersion hood and the substrate, etc. The determination of the interaction parameter can be performed using standard methodology or standard procedures. It can, e.g., be performed by a computing means, provided with suitable algorithms.

Repeating these experiments for different interaction times can allow obtaining information about the dynamic behaviour of the interaction between the immersion fluid 202 and the components of materials 210 in a region 208 of a substrate 212, i.e., the way the interaction varies with time. It is to be noted that, due to the low concentrations of diffused or leached components it is important to avoid cross-contamination in the experiments. Therefore optional intermediate cleaning steps are preferred. Such cleaning steps can comprise providing additional quantities of immersion fluid with an immersion hood or any other instruments to be used on substrates free of materials to be studied. Furthermore additional efforts can be performed for avoiding cross-contamination by non re-use of recipients, use of fluid dispensers with disposable tips, etc.

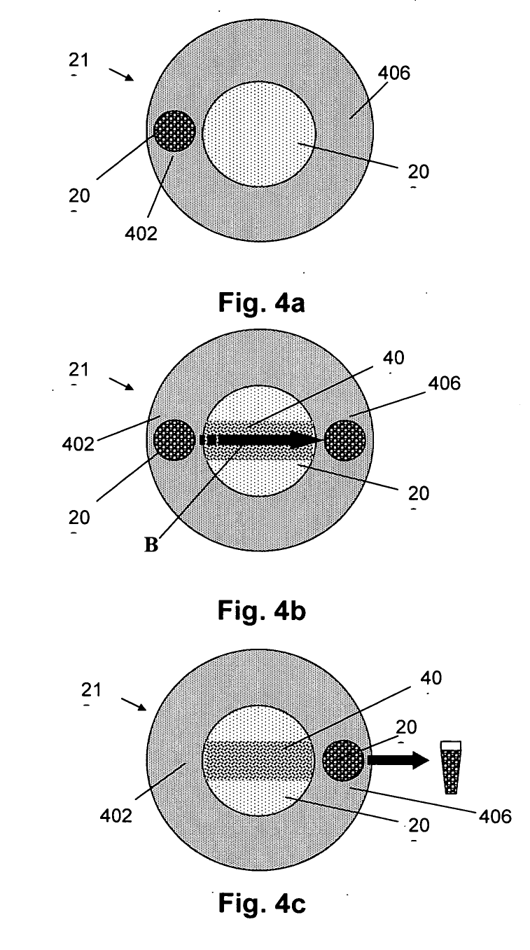

In a second aspect, a method is provided for studying interaction between an immersion fluid and materials present on a substrate suitable for lithographic processing as described in the first aspect, but wherein the step of providing a spatially controlled contact between the substrate and the immersion fluid by providing relative movement of the contact area between the substrate and the immersion fluid is obtained by providing a controlled spatial contact path between the substrate 212 and the immersion fluid 202. The controlled spatial contact path can be obtained using a set-up as shown in FIG. 2 or in FIG. 3. A preferred way of providing a controlled spatial contact path between the substrate 212 and the immersion fluid 202 is shown by the schematic illustrations in FIG. 4a to FIG. 4c. In the method illustrated in FIG. 4a to FIG. 4c, a substrate 212 is used wherein besides a first region 208 comprising materials 210 for which the interaction with the immersion fluid 202 is to be studied, the substrate 212 also comprises a second region 402 free of materials and/or components for which the interaction with the immersion fluid 202 is to be studied. For example, the substrate 212 can be a silicon substrate having, in a first region 208, at least a resist layer or a top coating such as, e.g., an anti-reflective top coating, whereas in a second region 402 no resist or top coating is present. The latter can, e.g., be obtained by either spatially selective providing the material 210 only in the first region 208 of the substrate 212 or by removing these materials 210 afterwards from the second region 402, e.g., by performing a large edge bead removal. Providing a controlled contact path then can be performed by first contacting the immersion fluid 202 with the substrate 212 in a second region 402, not comprising any component for which the interaction with the immersion fluid 202 is to be studied, as schematically shown in top view in FIG. 4a. During this contacting no leaching of or diffusion into materials for which the interaction is to be studied occurs. The latter may be preferred as the control on the contact time between the immersion fluid 202 and the substrate 212 when applying or removing the immersion fluid 202 may be difficult. Then a relative movement between the immersion fluid 202 and the first region 208 of the substrate 212 comprising the materials and/or components 210 for which the interaction is to be studied, is provided. The latter may be performed by moving either the immersion fluid 202 with respect to the substrate 212 or by moving the substrate 212, e.g., on a substrate stage, with respect to the tip 204 comprising the immersion fluid 202. Typically, for a fixed contact path 404 over the first region 208, the interaction time can be varied by altering the speed of relative movement. If, e.g., a fixed speed of movement is used, a well known contact time between the immersion fluid 202 and the materials and/or components 210 in the first region 208 can be determined. During this contact, interaction between the immersion fluid 202 and the materials and/or components 210 in the first region 208 may occur. The latter is schematically illustrated in FIG. 4b, indicating the relative movement by arrow B and indicating the contact path 404. The relative movement between the immersion fluid 202 and the substrate 212 is ended in a third region 406 of the substrate 212, i.e., also a region being free of materials and/or components for which interaction is to be studied. The third region 406 and the second region 402 can be the same region or can be separate and/or different regions. There, no interaction between the immersion fluid 202 and the materials and/or components 210 for which interaction is to be studied occurs anymore. The latter is advantageous as the control on the contact time between the immersion fluid 202 and the substrate 212 when applying or removing the immersion fluid 202 may be difficult. Thus, the path along which interaction may occur thus can be well controlled, i.e., any non-controllability in the removal phase of the immersion fluid does not influence the interaction time. The end phase of the movement is illustrated schematically in FIG. 4c. Such a method can advantageously be performed in an immersion lithography scanner or immersion lithography interference printer allowing movement of either one or both of the substrate or the immersion fluid, via movement of the immersion hood, with respect to each other. The total contact time between the immersion fluid and the materials to be studied typically is a function of contact area, scan speed and length of path followed.

In a third aspect, a method is provided for studying interaction between an immersion fluid and a substrate as described in any of the previous aspects, but wherein, for the step of determining an interaction parameter indicative of the interaction, a physical shape of the immersion fluid 202 at the contact interface between the immersion fluid 202 and the substrate 212 is taken into account. The shape of the immersion fluid 202 at the contact interface between the immersion fluid 202 and the substrate 212 typically is not a square or rectangle but has a droplet shape, such as a substantially round or elliptical shape. When providing a controlled contact using a dynamic interaction measurement as described in the previous embodiments, for a given direction of movement and a given movement rate, different contact times will occur at different positions in the path depending on the shape of the droplet. The shape of the interface during the contact can be fixed and can be taken into account. The latter can, e.g., be done based on a model taking into account the lyophilic/lyophobic, i.e., hydrophilic/hydrophobic, if the immersion fluid is water, behaviour of the substrate 212, the shape of the means providing relative motion for the contact area, such as, e.g., the shape of the tip or ring housing, and the volume of the immersion fluid 202 provided at the tip 204. In other words, in the preferred embodiments, variation of the contact time at different positions in the contact path 404 is taken into account by determining an average contact time between the immersion fluid 202 and the substrate 212 for the contact. Such an average contact time is determined not only based on the surface area of the contact path 404 on the substrate and a contact time, but also on the momentary shape or different momentary shapes of the immersion fluid 202 at the interface. This leads to a more accurate determination of the interaction parameter.

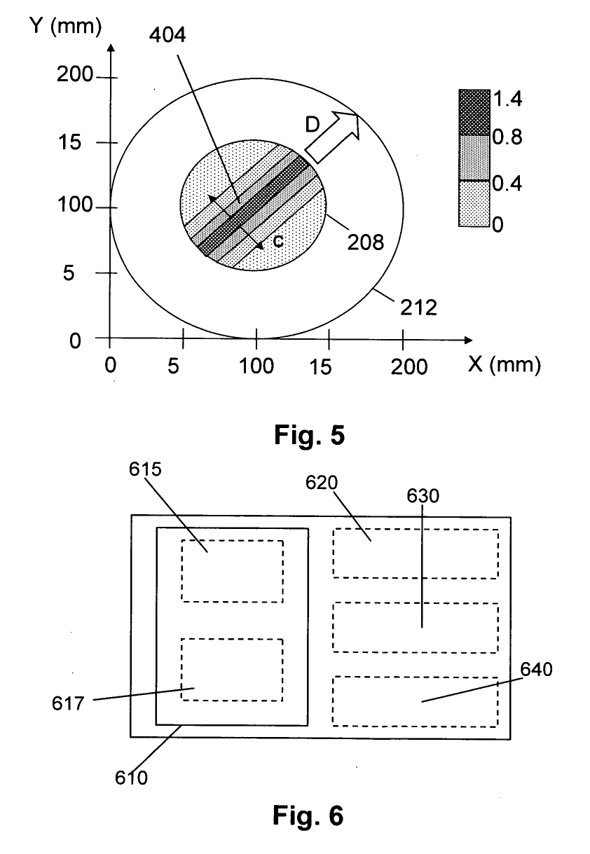

The latter is illustrated for a contact path 404 obtained by relative movement between the immersion fluid 202 and the substrate 212 in a dynamic interaction measurement. FIG. 5 indicates the soak time, i.e., the interaction time between the immersion fluid 202 and the substrate 212 for diagonal scanning at a speed of 24.7 mm/second for a rather elliptical interface shape of the immersion fluid 202. It can be seen, in the direction perpendicular to the scanning direction, indicated by arrow D, that the contact time is largest in the centre and smallest at the edges. Taking into account the shape of the immersion fluid 202 at the contact interface and the length of the contact path 404 an average soak time of 0.82 seconds is obtained.

In a fourth aspect, a method is provided for setting up a lithographic process. The method comprises selecting a number of system parameters and processing parameters in order to obtain an appropriate lithographic process. It is a specific feature of the preferred embodiments that at least one of the system parameters and/or at least one of the processing parameters are selected based on interaction properties between the immersion fluid 202 and the substrate 212 determined using a method according to any of the above described methods for studying interaction. The system parameters and/or the processing parameters can comprise for example any of the exposure conditions, the resist used, the presence and/or type of a top coat covering the resist, the type of immersion fluid used, the scan speed of a lithographic scanning system used, etc. As preferred embodiments allow accurate modelling of the interaction behaviour between the immersion fluid and materials in the substrate, fitting parameters of physical models can be calibrated for any substrate-immersion combination. When exposing a real chip this information can be used to calculate the effective interaction between the immersion fluid and the materials in the substrate, to determine the influence on the dimension control and hence to perform a correction. Such a correction can be a general correction, but also can be a die-by-die correction. As such the information obtained by this method allows obtaining an optimised lithographic process.

In a fifth aspect, a measurement kit is provided for studying the interaction between an immersion fluid and a substrate, e.g., a resist or top coat covering the resist. The measurement kit comprises a tool that typically is adapted for performing a method for studying the interaction by providing a spatially controlled contact between the substrate, or part thereof, and the immersion fluid by providing movement between the contact area between the substrate and the immersion fluid over the substrate. The method for studying the interaction also can comprise obtaining analysis results of at least one of the substrate and the immersion fluid which have been contacted with each other and determining an interaction parameter thereof. Such a tool can be a substrate with a first region comprising at least a first material for which one wants to study the interaction with immersion fluid and a second region being free of such materials or components thereof.

In a sixth aspect, a measurement tool is provided for studying the interaction between an immersion fluid and at least a first material in a substrate suitable for lithographic processing, the measurement tool comprising a means for providing a spatially controlled contact by providing a relative movement of a contact area between the substrate and the immersion fluid over the substrate. Such a measurement tool also can comprise a means for providing immersion fluid in contact with the substrate and for removing immersion fluid from the substrate. Such a system can comprise a substrate stage and a means for providing immersion fluid in contact with the substrate. In order to generate relative movement of the contact area between the substrate and the immersion fluid over the substrate, one of the substrate stage or the means for providing immersion fluid is to be moveable, e.g., a setup of at least one, preferably two, scanning stage can be provided, which is able to move, and a fluid head can be provided. Furthermore, an automated or manual fluid dispenser and retrieval system can be provided. Also a means for cleaning, such as, e.g., a means for providing programmable cleaning cycles, and a robot for substrate manipulation can be provided. A schematic representations of such a set-up is shown in FIG. 6, comprising a means for providing a spatially controlled contact 610 which itself can comprise a substrate stage 615 and a means for providing immersion fluid 617, at least one of these being moveable. Furthermore a means 620 for dispensing and retrieving the immersion fluid also can be provided. Optionally means for cleaning 630 and means for manipulating substrates 640 also can be provided. The substrate can be part of the tool itself. Furthermore, also more complicated systems, such as for example a system comprising a movable arm having a nozzle for providing a liquid, the moveable arm being adapted for moving over the substrate such that the liquid is in contact with the substrate, can be used.

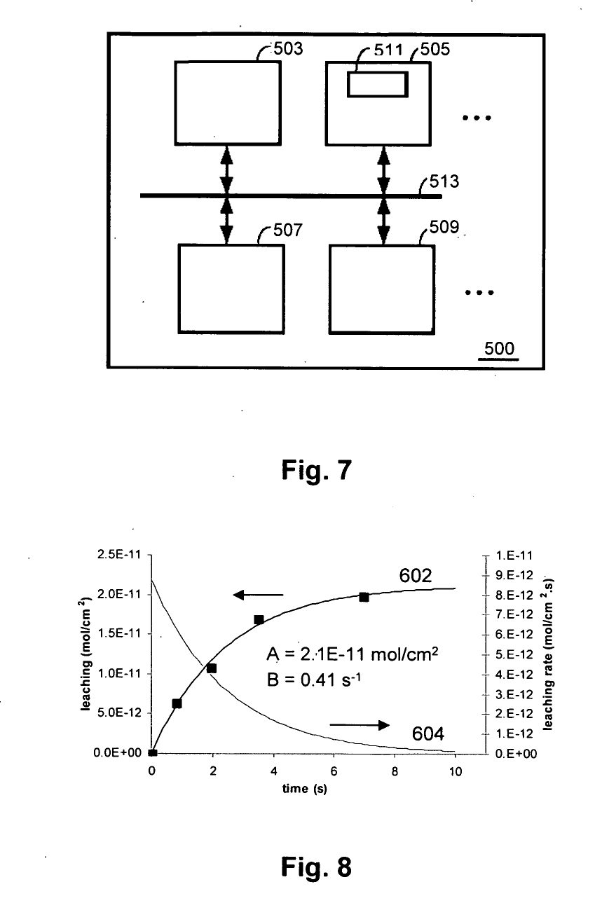

In a seventh aspect, a computing means is provided for deriving an interaction parameter for interaction between an immersion fluid and a substrate suitable for use in lithographic processing. The computing means therefore comprises a means adapted for obtaining information about a spatially controlled contacting step by providing a relative movement of the contact area between an immersion fluid and only part of a substrate and a means adapted for obtaining information about a quantitative analysis of a component of the immersion fluid in the substrate or of a component of the substrate in the immersion fluid. The computing means furthermore comprises a means adapted for deriving from the obtained information about a spatially controlled contacting step and the obtained information about the quantitative analysis, a value for an interaction parameter indicative of an interaction between the immersion fluid and the substrate. In one example, the information about a spatially controlled contacting step by providing a relative movement to the contact area between an immersion fluid and a substrate or part thereof can comprise a movement rate used for moving an immersion fluid over a substrate, size information about a region comprising a material for which interaction with the immersion fluid is to be studied and information about the immersion fluid, such as, e.g., a size of the contact interface and a volume of the immersion fluid used. The latter can allow, using the information about the quantitative analysis and using computational power, to derive leaching or dissolution rates and amounts for leaching of components of the substrate in the immersion fluid or dissolution of components of the immersion fluid in the substrate. An exemplary processing system 500 is shown in FIG. 7. FIG. 7 shows one configuration of processing system 500 that includes at least one programmable processor 503 coupled to a memory subsystem 505 that includes at least one form of memory, e.g., RAM, ROM, and so forth. A storage subsystem 507 can be included that has at least one disk drive and/or CD-ROM drive and/or DVD drive. In some implementations, a display system, a keyboard, and a pointing device can be included as part of a user interface subsystem 509 to provide for a user to manually input information. Ports for inputting and outputting data can also be included. More elements such as network connections, interfaces to various devices, and so forth, can be included, but are not illustrated in FIG. 7. The various elements of the processing system 500 can be coupled in various ways, including via a bus subsystem 513 shown in FIG. 7 for simplicity as a single bus, but will be understood to those in the art to include a system of at least one bus. The memory of the memory subsystem 505 can at some time hold part or all (in either case shown as 511) of a set of instructions that, when executed on the processing system 500, implement the step(s) of the method embodiments described herein. Thus, while a processing system 500 such as shown in FIG. 7 is prior art, a system that includes the instructions to implement aspects of the preferred embodiments is not prior art, and therefore FIG. 7 is not labelled as prior art.

It is to be noted that the processor 503 can be a general purpose, or a special purpose processor, and can be for inclusion in a device, e.g., a chip, that has other components that perform other functions. Thus, one or more preferred embodiments can be implemented in digital electronic circuitry, or in computer hardware, firmware, software, or in combinations of them. Furthermore, aspects of the preferred embodiments can be implemented in a computer program product tangibly embodied in a carrier medium carrying machine-readable code for execution by a programmable processor. Method steps of preferred embodiments can be performed by a programmable processor executing instructions to perform functions of those aspects of the preferred embodiments, e.g., by operating on input data and generating output data. Accordingly, the preferred embodiments include a computer program product which provides the functionality of any of the methods according to the preferred embodiments when executed on a computing device. Further, the preferred embodiments include a data carrier such as for example a CD-ROM or a diskette which stores the computer product in a machine-readable form and which executes at least one of the methods of the preferred embodiments when executed on a computing device. Nowadays, such software is often offered on the Internet or a company Intranet for download, hence the preferred embodiments include transmitting the computer product according to the preferred embodiments over a local or wide area network.

By way of example, the preferred embodiments not being limited thereto, experimental results are provided for the study of leaching of photo-acid generator (PAG) from resist. The substrates under test were 200 mm silicon wafers prepared using a priming, coating, baking, rinsing and edge cleaning process according to the TEL Act 8 track, as well known by a person skilled in the art. Leaching of the PAG anion of a 150 nm PAR817 resist layer, obtainable from Sumitomo Chemical Co, is studied in an immersion interference printer as available from ASML Wilton. A method according to the second aspect and FIG. 4a to 4c is used. The resist layer is applied to the silicon wafers using in-line dispensing. Quantitative concentration analysis is performed using mass spectrometry with atmospheric pressure electrospray ionisation, leading to a PAG anion detection limit of about 0.2 ppb, corresponding with a 3.10⁻¹⁴ mol/cm² concentration.

Additional precautions comprising cleaning steps with three water dispensings and retrievals on a non-coated substrate, the use of accurate 1000 μl Finn-pipettes and disposable tips for the pipettes and disposable sample recipients are taken to avoid cross-contamination between experiments. In the bare silicon regions of the resist coated samples, obtained by using an edge bead removal step of 50 mm, no detectable leaching is present. These regions are covered with a Hexamethyldisilazane (HMDS) primer, to make the surface hydrophobic, which is required for the immersion interference printer.

Using a scanning method as described in the second aspect, contact or interaction times between 0.82 s and 6.9 s are obtained. The latter is within the relevant range for contact times in high volume manufacturing immersion scanners. The shortest contact times thereby are obtained by diagonally scanning using both a horizontal and vertical stage of the immersion interference printer as the latter allows obtaining the highest speed. The total contact time typically is a function of contact area, scan speed and length of path followed. A volume of 2 ml of the immersion fluid, being water in the present example, is applied, whereby 1 ml immersion fluid is recovered for analysis. For analysis and determination of the interaction parameter, it is assumed that perfect mixing of the immersion fluid and the leached component occurs and furthermore the shape of the immersion fluid at the interface during the contact is taken into account to obtain an average interaction time. The latter is obtained using dedicated software.

For analysis of the effect of exposure on leaching, open frame exposures on an ASML PASS5500/1100 dry 193 nm scanner are used followed by the normal leaching procedure.

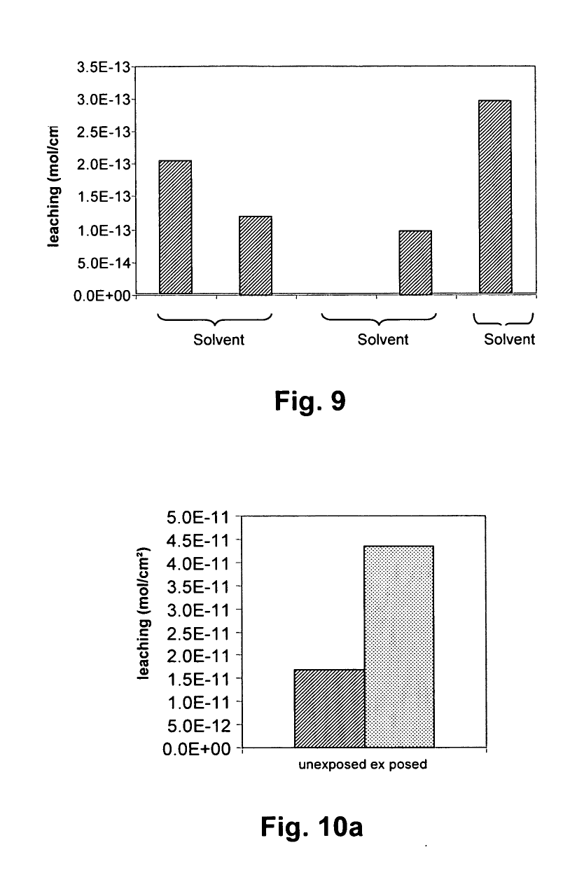

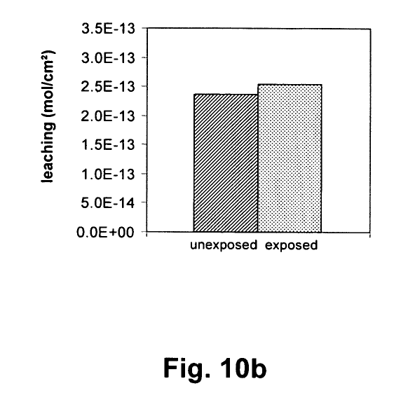

Results for the dynamic leaching of PAG for an unexposed PAR-817 resist coating are shown in FIG. 8. The amount of leached component per unit of surface contact area, expressed in mol/cm², is shown as a function of time. The experimental results are indicated using squares. As expected, it can be seen that the amount of leached component per unit of surface contact area saturates within a few seconds. It also can be seen that the amount of leached component per unit of surface interaction at interaction times between 1 and 2 seconds, which are typical interaction times during standard lithographic processing of substrates, differs substantially from this saturation limit. The results are in good agreement with the two parameter model for leaching as described in R. R. Dammel et al. SPIE Proceedings Vol. 7573 (2005) pp 95-101. The two parameter model is described by equations [1] and [2]
A-A.exp(−Bt) [1]
A.B.exp(−B.t) [2]
indicating the total amount of leached component in time t and the leaching rate respectively. A (in mol/cm²) thereby gives the saturation level and B (in s⁻¹) is a time constant. The larger B, the quicker the saturation level is reached. In FIG. 8, equation [1] is fitted to the experimental results, shown by curve 602. Curve 604 indicates the corresponding leaching rate. From the A and B parameters that have been obtained from the fit, the leaching rate at any point in time can be calculated. The leaching rate at t=0, i.e., the value for A*B, is 8.8*10⁻¹² mol/cm²·s and the leached amount in the first second is 7.2*10⁻¹² mol/cm². The high leaching levels obtained are not surprisingly, as PAR-817 is developed for dry lithography, not for immersion lithography. In further experiments leaching of a PAG anion in PAR-817 was studied as a function of different parameters of the lithographic processing. Experiments are performed for a PAR-817 resist obtainable from Sumitomo Chemical Co and for a PAR-817 resist covered with a TCX-007 top coat obtainable from JSR Corporation. The amount of leaching as a function of the solvent used is shown in FIG. 9 for different experiments. It can be seen that different solvents may result in different amounts of leaching. FIG. 8 illustrates the effect of another lithographic processing parameter, i.e., exposure, for leaching of a PAG anion in PAR-817 with and without a TCX-007 top coat. It can be seen that a flood exposure has little effect on leaching for a PAR-817 resist with top coat, but that the flood exposure significantly effects leaching in PAR-817 without top coat. The latter is illustrated in FIG. 10a and FIG. 10b showing the leaching effects for resist PAR-817 and for resist PAR-817 with topcoat respectively for both exposed and unexposed substrates with a 25 mJ flood exposure.

From the above examples, it can be seen that the leaching measurements can be used for optimising the lithographic processing of a substrate.

It is an advantage of preferred embodiments that the amount of immersion fluid used for contacting the substrate can be relatively low, thus increasing to a substantially high component/immersion fluid ratio leading to an increase of the detection limit and an increase in measurement accuracy.

All references cited herein are incorporated herein by reference in their entirety. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

The above description provides several methods and materials of the present invention. It is to be understood that although preferred embodiments, specific constructions and configurations, as well as materials, have been discussed herein for devices according to the present invention, various changes or modifications in form and detail may be made without departing from the scope and spirit of this invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention as embodied in the attached claims.

What is claimed is:

1. A method for studying an interaction between an immersion fluid and a first material in a substrate configured for lithographic processing, the method comprising:
providing a spatially controlled contact by providing a relative movement of a contact area between the substrate and the immersion fluid over the substrate using a relative movement between an immersion hood and the substrate; thereafter
obtaining an analysis result of at least one of the immersion fluid and the first material in the substrate; and,
determining from the analysis result an interaction parameter indicative of an interaction between the immersion fluid and the first material in the substrate;
wherein the method comprises, prior to providing a spatially controlled contact between the immersion fluid and the substrate, providing a volume of the immersion fluid to the immersion hood.

2. The method according to claim 1, wherein the first material comprises first components, wherein the step of determining an interaction parameter comprises determining an interaction parameter indicative of leaching from at least one of the first components of the first material in the immersion fluid.

3. The method according to claim 2, wherein the first material is selected from the group consisting of a photoresist and a top anti-reflective coating.

4. The method according claim 1, wherein a physical size of the contact area is substantially constant during the step of providing a spatially controlled contact.

5. The method according to claim 1, further comprising, prior to providing a spatially controlled contact, providing the substrate in an immersion lithography processing system.

6. The method according to claim 1, wherein providing a relative movement of the contact area comprises providing a contact path by providing a relative movement between the immersion fluid and a first region of the substrate parallel to the substrate.

7. The method according to claim 6, wherein the substrate further comprises a second region, the second region being free from materials comprising the first components, and wherein providing a contact path comprises:
initiating a contact path between the immersion fluid and the substrate in the second region; thereafter
contacting the immersion fluid with the first region; and thereafter
ending the contact path between the immersion fluid and the substrate in the second region.

8. The method according to claim 6, wherein the interaction parameter between the immersion fluid and the first material in a first region of the substrate is selected by selecting a parameter selected from the group consisting of a speed of relative movement and a contact path length.

9. The method according to claim 8, wherein obtaining analysis results of at least one of the immersion fluid and the first material in the substrate comprises using a technique selected from the group consisting of mass spectroscopy, scanning electrochemical microscopy, and liquid scintillation counting.

10. A method for setting up lithographic processing of a substrate, the method comprising selecting at least one item selected from the group consisting of an immersion fluid, a substrate, a system parameter of a lithographic processing system, and a processing parameter of a lithographic process, wherein the selecting is based on a dynamic interaction result between an immersion fluid and a first material in a substrate, as obtained by a method according to claim 1.

11. A measurement tool for studying an interaction between an immersion fluid and a first material in a substrate configured for lithographic processing, the measurement tool comprising a means for providing a spatially controlled contact by providing a relative movement of a contact area between the substrate and the immersion fluid over the substrate, wherein the measurement tool further comprises a means for providing a volume of immersion fluid to an immersion hood prior to a spatially controlled contact between the immersion fluid and the substrate.

12. A measurement kit for studying an interaction between an immersion fluid and at least a first material in a substrate configured for lithographic processing according to the method of claim 1, the measurement kit comprising:
the substrate comprising a first region comprising at least the first material configured for lithographic processing, the first material comprising first components, and the substrate comprising a second region being free of materials comprising the first components.

13. A computer program product for deriving an interaction parameter for an interaction between an immersion fluid and a first material in a substrate, wherein the computer program product is configured to:
obtain information about a spatially controlled contacting step between the immersion fluid and only a part of the substrate;
obtain information about a quantitative analysis of a component of the immersion fluid in the substrate or of a component of the substrate in the immersion fluid; and
derive from the obtained information a value for an interaction parameter indicative of an interaction between the immersion fluid and the first material in the substrate,
wherein obtaining information comprises obtaining information about a spatially controlled contacting step between the immersion fluid and only a part of the substrate for a volume of immersion fluid provided to an immersion hood prior to the spatially controlled contact.
